(19) United States
(12) Patent Application Publication
(10) Pub. No.: US 2011/0136898 A1
(43) Pub. Date: Jun. 9, 2011

(54) TREATMENT OF RETINAL DEGENERATION

(75) Inventors: Thomas Cotter, Co. Cork (IE); Francesca Doonan, Cork (IE); Nuria Sanvicens Diez, Barcelona (ES); Carolyn O' Driscoll, Cork (IE); Angel Ramon Messeguer Peypoch, Barcelona (ES)

(73) Assignees: UNIVERSITY COLLEGE CORK, NATIONAL UNIVERSITY OF IRELAND, CORK, Co. Cork (IE); CONSEJO SUPERIOR DE INVESTIGACIONES CIENTIFICAS, Madrid (ES)

(21) Appl. No.: 13/057,548

(22) PCT Filed: Aug. 5, 2009
(86) PCT No.: PCT/IE2009/000055

§ 371 Date: Feb. 4, 2011

(30) Foreign Application Priority Data
Aug. 5, 2008 (IE) S2008/0647

(51) Int. Cl. A61K 31/353; A61P 27/02

(52) U.S. Cl. 514/456

(57) ABSTRACT

The use of a compound of general formula (I): or a pharmaceutically acceptable salt thereof, in the manufacture of a medicament for the treatment or prevention of a disease or condition characterised by apoptosis or degeneration of mammalian cells, wherein: R1 is a alkoxy, alkyl, ether or ester group; R2 is H or has the formula wherein Y is linear or branched, saturated or unsaturated, aliphatic group with from 2 to 23 carbon atoms, or a cyclic group, and which can contain substituents selected from the group consisting of hydroxyl, alkoxy, amino, carboxyl, cyano, nitro, alkylsuphonyl or halogen atoms, X is O or S; and R3 is any substituent.

### TECHNICAL FIELD

The invention relates to a method of treating or preventing a disease or condition characterised by apoptosis or degeneration of mammalian cells, especially retinal photoreceptive cells.

### BACKGROUND TO THE INVENTION

The loss of retinal cells in Age-related Macular Degeneration (AMD) and Glaucoma are the two leading causes of blindness in the developed worked. Retinitis Pigmentosa (RP) is a rarer related condition that also leads to loss of sight. RP is a group of hereditary disorders of the retina caused by mutations in numerous genes involved in photoreceptor structure or function. The disease is characterized by early loss of photoreceptors leading to blindness. Glaucoma is caused by a number of different pathological mechanisms that in most cases result in elevated intraocular pressure (IOP) within the eye. Like RP, it is a multiple gene-related disease and genetic factors play a complex role in glaucoma predisposition. Over time, the increase in IOP causes damage to the optic nerve and gradual and continuous loss of retinal ganglion cells. Cell loss in AMD (Age-related Macular Degeneration) also occurs as a result of apoptosis of retinal pigment epithelial (RPE) cells followed by apoptosis of photoreceptors. A central feature of each of the above diseases is that retinal cell loss occurs by a cell death process known as apoptosis.

There are no drugs on the market for the treatment of RP. There are a small number in early stage development, but most of them rely on repeated intavitreal injections directly into the eye for their effect or a gene therapy approach. For RP, there is a market realisation that preventing apoptosis may be a treatment option. In the case of glaucoma there are several treatment options currently available for this very large market segment. Most of these rely on the delivery of the drug in the form of eye drops. The goal of such treatments is to reduce the intra ocular pressure that is a causative factor that leads to the loss of retinal ganglion cells. Like RP there are no current treatments focused on the prevention of retinal ganglion cell apoptosis. Finally, for AMD of which there are two main forms there are a number of treatment options ranging from laser therapy to the use of inhibitors that prevent blood vessel proliferation which is a characteristic of the condition and leads to loss of photoreceptor cells. Again each of these treatment options is invasive and requires repeated hospital visits.

It is an object of the invention to overcome at least one of the above problems.

### BRIEF SUMMARY OF THE INVENTION

Accordingly, the invention relates to a method of treating or preventing a disease or condition characterised by apoptosis or degeneration of mammalian cells, especially retinal photoreceptive cells. The method of the invention comprises a step of treating an individual with a therapeutically effective amount of a compound of general formula (I)

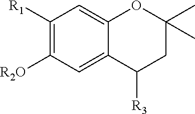

or a pharmaceutically acceptable salt thereof, wherein:

R1 is a alkoxy, alkyl, ether or ester group;

R2 is H or has the formula

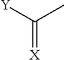

wherein Y is linear or branched, saturated or unsaturated, aliphatic group with from 2 to 23 carbon atoms, or a cyclic group, and which can contain substituents selected from the group consisting of hydroxyl, alkoxy, amino, carboxyl, cyano, nitro, alkylsuphonyl or halogen atoms, X is O or S; and R3 is any substituent (hereafter “Active”).

In a preferred embodiment of the invention, R1 is a methoxy group, and R2 is typically H.

In one embodiment, the Active is a compound of general formula (II),

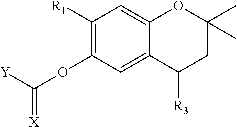

or a pharmaceutically acceptable salt thereof, in which: Y is linear or branched, saturated or unsaturated, aliphatic group with from 2 to 23 carbon atoms, or a cyclic group, and which can contain substituents selected from the group consisting of hydroxyl, alkoxy, amino, carboxyl, cyano, nitro, alkylsuphonyl or halogen atoms; X is O or S; and R3 is any substituent.

Suitably,

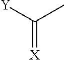

is selected from the group consisting of tert-butanoyl, hexanoyl, 2-ethylhexanoyl, octanoyl, decanoyl, lauroyl, myristoyl, palmitoyl, stearoyl, oleoyl, or lineoyl.

Typically, Y is an alicyclic group, or an aromatic cyclic group, or a heterocyclic group.

In one embodiment,

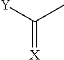

is selected from the group consisting of —CO—(CH₂)₀₋₆phenyl, —CO—(CH₂)₀₋₆(1-napthyl), —CO—(CH₂)₀₋₆(2-napthyl), —CO—(CH₂)₀₋₆CH(phenyl)₂, —CO-(2-fluorophenyl), —CO-cyclohexyl, α-lipoyl, L-prolyl, D-prolyl, biotinyl-CO-(4-imidazolyl), —CO-(2-pyridyl), —CO-(2-thienyl), —CO-(2-furyl), —CO-(3-furyl).

In a preferred embodiment of the invention, X is O.

In one embodiment, R1 is a methoxy group and OR₂ is a hydroxyl group (BP—FIG. 1A).

In another embodiment, R₁ is a methoxy group and OR₂ is an acetate ester (Derivative BP-1—FIG. 1B).

In another embodiment, R₁ is a methoxy group and OR₂ is a pivalate ester (Derivative BP-2—FIG. 1C).

In one embodiment, R₁ is a methoxy group and OR₂ is a laureate ester (Derivative BP-4—FIG. 1E).

In another embodiment, R₁ is a methoxy group and OR₂ is a 2-methylhexanate ester (Derivative BP-3—FIG. 1D).

In one embodiment, R₁ is a methoxy group and OR₂ is a phenyl ester (Derivative BP-5—FIG. 1F).

In another embodiment, R₁ is a methoxy group and OR₂ is a o-fluorophenyl ester (Derivative BP-6—FIG. 1G).

In one preferred embodiment, the Active is 3,4-dihydro-6-hydroxy-7-methoxy-2,2-dimethyl-1(2H)-benzopyran (BP).

In another embodiment, the Active is a compound of general formula (III),

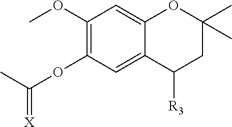

or a pharmaceutically acceptable salt thereof, in which X is O or S, and R3 is any substituent.

In another embodiment, the Active is a compound of general formula (IV),

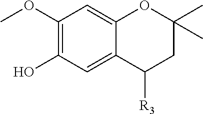

or a pharmaceutically acceptable salt thereof, in which R3 is any substituent.

Typically R3 is selected from the group consisting of: H; halogen; lower alkyl; lower alkoxy; hydroxyl; amine; thiol; NHR4; or a substituted or unsubstituted aromatic ring structure in which the substituents (if included) are selected from the groups consisting of H, halogen, lower alkyl, lower alkoxy, hydroxyl, amine, and thiol, and wherein R4 is any substituent. Suitably, R4 is selected from the group consisting of: halogen; lower alkyl; lower alkoxy; hydroxyl; amine; thiol; NHR4; or a substituted or unsubstituted aromatic ring structure in which the substituents (if included) are selected from the groups consisting of H, halogen, lower alkyl, lower alkoxy, hydroxyl, amine, and thiol.

In a preferred embodiment, R3 and R4 are, independently, C4 to C8 straight alkyl chains, preferably a C5 to C7 straight alkyl chain, and ideally a C6 straight alkyl chain.

In one embodiment, the Active is selected from the group consisting of:

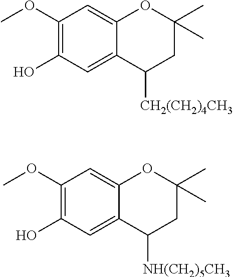

The Active is administered in a therapeutically effective amount to treat or prevent the disease or condition. When the invention relates to therapy, as opposed to prophylaxis, the individual is generally one in need of such treatment such as a patient having a retinal degenerative condition. Suitably, the disease or condition is an retinal degenerative disease, such as, for example, Retinitis Pigmentosa (RP), Glaucoma, or Age-related Macular Degeneration (AMD). In an alternative embodiment, the disease or condition is a mammalian degenerative disease, such as a neurodegenerative disease.

The invention also relates to the use of the Active as a medicament. Suitably, the medicament is for treating a retinal degenerative disease, especially Retinitis Pigmentosa (RP), Glaucoma, or Age-related Macular Degeneration (AMD).

The invention also relates to the use of the Active in the manufacture of a medicament for the treatment or prevention of a disease or condition characterised by apoptosis or degeneration of mammalian cells. In particular, the invention relates to the use of the

Active in the manufacture of a medicament for the treatment or prevention of an retinal degenerative condition such as Retinitis Pigmentosa (RP), Glaucoma, or Age-related Macular Degeneration (AMD).

The invention also relates to the Active compounds of Formula (I), (II) or (III), or pharmaceutically acceptable salts thereof. The invention also relates to the Active compound of FIG. 1D, or pharmaceutically acceptable salts thereof.

The invention also relates to a pharmaceutical formulation comprising an Active compound of the invention, in combination with a suitable pharmaceutical excipient. The invention also relates to the use of an Active compound of the invention as a medicament.

### BRIEF DESCRIPTION OF THE FIGURES

FIG. 1: Shows the chemical structure of BP and BP derivatives, including BP (FIG. 1A), BP-1 (FIG. 1B), BP-2 (FIG. 1C), BP-3 (FIG. 1D), BP-4 (FIG. 1E), BP-5 (FIG. 1F), BP-6 (FIG. 1G), BP-X (FIG. 1H), and BP-Y (FIG. 1I).

FIG. 2: Is a histogram of results showing the effectiveness of a 25 μM concentration of each of the BP derivatives BP-1 to BP6 against increasing concentrations of SNP in photoreceptor cells. Error bars are +/−standard deviation-SD.

FIG. 3: Is a histogram showing the scavenging ability of each of the BP derivatives BP-1 to BP6 at 25 μM in photoreceptor cells in the presence of increasing concentrations of SNP. Error bars are +/−SD.

FIG. 4: Is a histogram of % protection afforded by a 25 μM concentration of each BP derivative BP1 to BP6 in photoreceptor cells as a function of the lipophilicity of each derivative. (P is a logarithmic value that indicates the lipophilicity of a compound).

While BP4 appears to have the greater protective capacity this derivative is very lipophilic rendering it problematic for administration. It is also a highly unstable compound.

Lipophilicity of the compounds is as follows: BP1<BP2<BP3=BP5=BP6<BP4

With lower lipophilicity, BP3 is far more stable than BP4, and hence the chosen derivative.

FIGS. 5 and 6: These figures show that both BP and BP3 protect photoreceptor cells from SNP induced apoptosis. Both BP and BP3 are effective at 25 μM but BP3 is equally as effective at a concentration 10 times lower than BP.

FIG. 7: In this figure the study was extended from ex vivo cultures to the light damage model in vivo. In this acute model of retinal disease albino balb/c mice are exposed to excessive white light causing the photoreceptor cells to die by apoptosis. This figure shows that mice exposed to bright white light undergo extensive apoptosis at 24, 48 and 72 hours post light damage. 1 hour pre-treatment with 200 mg/kg BP3 protects from the retinal damage observed. The protection extends to 48 hours with only a single injection of BP3. Administration of a second dose, or a ‘top up’ of 200 mg/kg BP3 at 24 hours post light damage affords significant protection at 72 hours.

FIG. 8 is a histogram of the results from FIG. 7. TUNEL positive cells in three independent retinae were counted. Counts were performed on the central 40× field of the outer nuclear layer (ONL) and graphed with error bars (+/−standard deviation-SD). The graph demonstrates approx 40% protection from light induced cell death in the central retina at 24, 48 and 72 hours.

FIG. 9A shows the results obtained in a chronic model of retinal degeneration called the rd10 model. In this model photoreceptor cells degenerate more slowly than the light model, over weeks rather than hours to days. Cell death is evident from postnatal day 18 (P18) and by P25 a significant loss of photoreceptor cell layers is observed. FIG. 9 A shows protection from photoreceptor cell death in the ONL when mice are injected daily with 200 mg/kg BP3. In this model, the central and peripheral retina degenerate at different rates. The loss of photoreceptors is greater in the central retina than the periphery between P18 and P25.

FIG. 9B, a histogram of the data shown in FIG. 9A, show that both the central and peripheral (divided into inferior and superior regions as illustrated by the schematic) areas of the retina are protected. Furthermore FIG. 9B shows that BP3 injection on alternate days affords the same level of protection as a daily injection regimen in all retinal areas.

FIG. 10: The ONL comprises two types of cell; rod photoreceptors and cone photoreceptors. FIG. 10 identifies cell types which are protected by BP3 treatment when administered on alternate days from P18 to P25. Rhodopsin is a protein specifically found in the outer segments (OS) of rod photoreceptors. Immunofluorescent staining using an antibody specific to rhodopsin indicates that more rhodopsin positive cells remain in the ONL of BP3 treated mice compared to vehicle. Peanut agglutinin (PNA) is a lectin which binds to carbohydrates found in the outer membrane of cone photoreceptors but which are absent from rods. Staining of retinal sections with PNA indicates that more cone cells are found in the ONL following BP3 treatment. Therefore the cells of the ONL involved in both colour and black/white vision are protected in the rd10 model by BP3.

FIG. 11 shows the results obtained in an acute model of Glaucoma: NMDA-induced excitotoxicity. In this model intravitreal injection of 40 mM NMDA results in the progressive loss of ganglion cells from the ganglion cell layer (GCL), accompanied by a thinning of the inner plexiform layer (IPL) over time. FIG. 11 shows the loss of cells in the GCL and the reduced IPL at 48 and 72 hours post insult. Administration of 200 mg/kg BP3 1 hour prior to NMDA injection significantly attenuates both ganglion cell loss and IPL thinning.

FIG. 12 shows the onset of apoptotic cell death in the GCL at 4 hours post NMDA injection. Ganglion cell death is significantly greater by 24 hours post damage at which point, cells of the inner nuclear layer (INL) also undergo cell death. By 48 and 72 hours post excitotoxicity the eventual loss of cells of the ONL is evident. Intraperitoneal (I.P.) injection of 200 mg/kg BP3 1 hour prior to NMDA injection significantly protects retinal cells in all layers at all time points examined, indicating universal protection by BP3 in the retina.

FIG. 13 is a histogram of the data from FIG. 12. TUNEL positive cells were counted across all the retinal layers from the inferior through the central to the periphery at the indicated timepoints. The graph indicates significant protection from NMDA-induced cell death at 4, 24, 48 and 72 hours.

### DETAILED DESCRIPTION OF THE INVENTION

The therapeutic method, and therapeutic products, of the invention are directed against diseases or conditions characterised by apoptosis or degeneration of mammalian cells. In one embodiment of the invention, the disease or condition characterised by apoptosis or degeneration of mammalian cells is an ocular disease or condition, especially a retinal degenerative condition or disease. The invention is particularly applicable for the treatment/prevention of retinal dystrophies. In one embodiment of the invention, the disease or condition characterised by apoptosis or degeneration of mammalian cells, is a neurodegenerative disease. Typically, the neurodegenerative disease is selected from the group comprising: motor neurone disease (ALS) or variants thereof including primary lateral sclerosis and spinal muscular atrophy; prion disease; Huntington's disease; Parkinson's disease; Parkinson's plus; Tauopathies; Chromosome 17 dementias; Alzheimer's disease; Multiple sclerosis (MS); hereditary neuropathies; and diseases involving cerebellar degeneration.

In a preferred embodiment of the invention, the retinal degenerative condition is selected from the group comprising: RP; Glaucoma; retinopathies; and AMD.

“Lower alkyl” means an alkyl group, as defined below, but having from one to ten carbons, more preferable from one to six carbon atoms (eg. “C—C-alkyl”) in its backbone structure. “Alkyl” refers to a group containing from 1 to 8 carbon atoms and may be straight chained or branched. An alkyl group is an optionally substituted straight, branched or cyclic saturated hydrocarbon group. When substituted, alkyl groups may be substituted with up to four substituent groups, at any available point of attachment. When the alkyl group is said to be substituted with an alkyl group, this is used interchangeably with “branched alkyl group”. Exemplary unsubstituted such groups include methyl, ethyl, propyl, isopropyl, a-butyl, isobutyl, pentyl, hexyl, isohexyl, 4,4-dimethylpentyl, octyl, 2,2,4-trimethylpentyl, nonyl, decyl, undecyl, dodecyl, and the like. Examplary substituents may include but are not limited to one or more of the following groups: halo (such as F, CI, Br, I), Haloalkyl (such as CC13 or CF13), alkoxy, alkylthio, hydroxyl, carboxy (—COOH), alkyloxycarbonyl (—C(O)R), alkylcarbonyloxy (—OCOR), amino (—NH2), carbamoyl (—NHCOOR— or —OCONHR), urea (—NHCONHR—) or thiol (—SH). Alkyl groups as defined may also comprise one or more carbon double bonds or one or more carbon to carbon triple bonds.

“Lower alkoxy” refers to O-alkyl groups, wherein alkyl is as defined hereinabove. The alkoxy group is bonded to the core compound through the oxygen bridge. The alkoxy group may be straight-chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy.

“Halogen” means the non-metal elements of Group 17 of the periodic table, namely bromine, chlorine, fluorine, iodine and astatine.

“Salt” is a pharmaceutically acceptable salt and can include acid addition salts such as the hydrochlorides, hydrobromides, phosphates, sulphates, hydrogen sulphates, alkylsulphates, arylsulphonates, acetates, benzoates, citrates, maleates, fumarates, succinates, lactates, and tartrates; alkali metal cations such as Na, K, Li; alkali earth metal salts such as Mg or Ca; or organic amine salts. Exemplary organic amine salts are tromethamine (TRIS) salts and amino acid salts (e.g. histidine salts) of the compounds of the invention.

In this specification the term “therapeutically effective amount” should be taken to mean an amount which results in a clinically significant reduction of degeneration or aptosis in cells having a phenotype characteristic of a degenerative condition (i.e. retinal photoreceptor cells from a patient with a retinal dystrophy, for example AMD or RP. Suitably, the Active is administered at a dose of between 1 microgram and 10 miligrams per ml, preferably between 10 micrograms and 5 miligrams per ml, more preferably between 100 micrograms and 2 miligrams per ml. Typically, it is given as a bolus dose. However, when continuous infusion is used, such as by intrathecal pump, the Active may be administered at a dosage rate of between 5 and 20 μg/kg/minute, preferably between 7 and 15 μg/kg/minute. In the context of the therapeutic aspects of the present invention, the term “individual in need thereof” shall be taken to mean an individual who is afflicted with a disease or condition which involves apoptosis or degeneration of mammalian cells, especially apoptosis or degeneration of the photoreceptive cell. Retinal degenerative conditions or diseases such as RP, Glaucoma, Retinopathies, and AMD, and variants thereof as described herein, are examples of such diseases or conditions.

In one embodiment of the invention, an individual in treated with the Active by direct delivery of the Active by a means selected from the group: intravenous delivery; intraperitoneal delivery; oral delivery; intramuscular delivery; intrathecal delivery; and inhaled delivery. Methods for achieving these means of delivery will be well known to those skilled in the art of drug delivery. Specific examples are provided below:
Delivered intrathecially by mini-osmotoc pump. (ref: Ignacio et al., Ann. N.Y. Acad. Sci. 2005, 1053: 121-136).
Intramuscular—delivery directly into muscle(s) by syringe or mini osmotic pump (Azzouz et al., Nat Med. 2005; 11(4):429-33).
Intraperitoneal—for systemic administration. Directly administered to peritoneum by syringe or mini osmotic pump (Kieran et al., Nat Med 2004; 10(4):402).
Subcutaneous—for systemic administration. Directly administered below the skin by syringe (Reinholz et al., Exp Neurol. 1999; 159(1):204-16).
Intraventricular—direct administration to the ventricles in the brain, by injection or using small catheter attached to an osmotic pump.(Sathasivam et al., 2005 Neuropath App Neurobiol; 31(5): 467)
Implant—Active can be prepared in an implant (eg small silicon implant) that will release the active. Implant can be placed at muscles or directly onto the spinal cord (Kieran and Greensmith, 2004 Neurosci 125(2):427-39).

In a particularly preferred embodiment of the invention, in which the indication is a retinal dystrophy, the active may be administered by direct intraocular or intravitreal injection, by topical application by means of eye drops, or by oral gavage.

In one embodiment of the therapy of the invention, the Active is linked to a coupling partner, e.g. an effector molecule, a label, a drug, a toxin and/or a carrier or transport molecule. Techniques for coupling the Active of the invention to both peptidyl and non-peptidyl coupling partners are well known in the art.

The invention provides methods of treatment and prevention of diseases or conditions characterized by apoptosis or degeneration of mammalian cells, especially photoreceptive cells, by administration to a subject in need of such treatment of a therapeutically or prophylactically effective amount of the Active. The subject is preferably an animal, including, but not limited to, animals such as monkeys, cows, pigs, horses, chickens, cats, dogs, etc., and is preferably a mammal, and most preferably human.

Apart from the specific delivery systems embodied below, various delivery systems are known and can be used to administer the Active of the invention, e.g., encapsulation in liposomes, microparticles, microcapsules. Methods of introduction include but are not limited to intradermal, intramuscular, intraperitoneal, intravenous, subcutaneous, intranasal, epidural, and oral routes. The Active may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the Active of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the Active of the invention locally to the area in need of treatment; this may be achieved, for example, by means of eye drops, intraocular injection, or an implant, said implant being of a porous, non-porous, or gelatinous material, including membranes, such as sialastic membranes, or fibers.

In another embodiment, the Active can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the Active can be delivered in a controlled release system. In one embodiment, a pump may be used (see Langer, supra; Sefton, CRC Crit. Ref. Biomed., Eng. 14:201 (1987); Buchwald et al., Surgery 88:75 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

The present invention also provides pharmaceutical compositions comprising the Active. Such compositions comprise a therapeutically effective amount of the Active, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the Active is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Active of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Active of the invention which will be effective in the treatment of a particular disorder or condition will depend on the nature of the disorder or condition, and can be determined by standard clinical techniques. In addition, in vivo and/or in vitro assays may optionally be employed to help predict optimal dosage ranges. The precise dose to be employed in the formulation will also depend on the route of administration, and the seriousness of the disease or disorder, and should be decided according to the judgment of the practitioner and each patient's circumstances.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

It has been demonstrated that there is a linear relationship between blood brain barrier (BBB) permeability and lipid solubility providing the MW of the molecule is under a 400-600 Da threshold. However, the presence of a hydroxyl group in the chemical structure of BP can significantly reduce its permeation through biological barriers. In the present Application, two approaches have been applied to increase the lipid solubility of the lead compound BP, namely (a) block the hydroxyl (R′) group by transforming it into an ester group or (b) substitute the methoxy residue (R) in BP for an alkoxy group containing a higher number of methylene groups. Six ester derivatives (BP1-6) of BP were synthesised using a parallel synthetic approach (BP is the common starting material of all the reactions). Optimizing the size of the side chain is an important consideration. If it's too large the compound can be sequestered by fatty tissue and may not reach its target. If it is too small the compound loses the ability to cross membranes and may be quickly excreted. The compounds synthesized using the first strategy included the acetate (BP-1), the pivalate (BP-2) and the laureate (BP-4) esters of the lead compound BP, two aromatic derivatives: the phenyl (BP-5) and o-fluophenyl (BP-6) esters and one α-substituted compound: the 2-methylhexanate ester (BP-3) of the lead compound BP.

Results shown below from retinal cells and retinal explants indicate that the BP-3 performs very effectively, inhibiting apoptosis both in retinal cells and explants. This suggests that BP-3 has greater lipophilicity than the lead compound BP, improving its bioavailability and allowing more of the compound to access the cell compartment.

### Experimental

Synthesis of 3,4-dihydro-6-hydroxy-7-methoxy-2,2-dimethyl-1(2H)-benzopyran (BP) derivatives

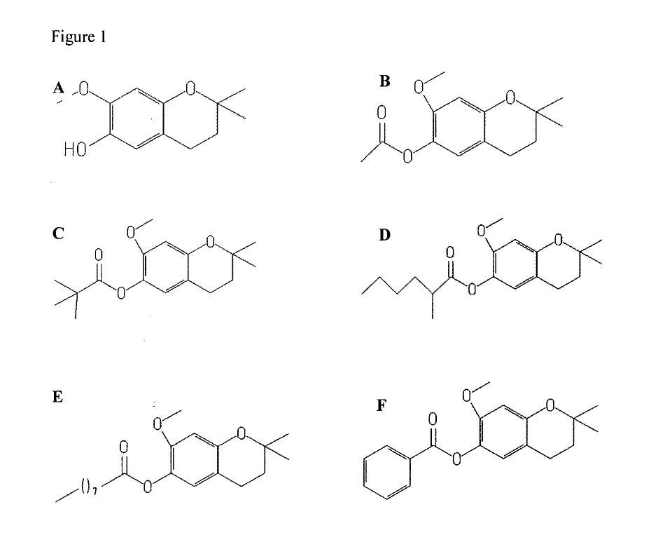

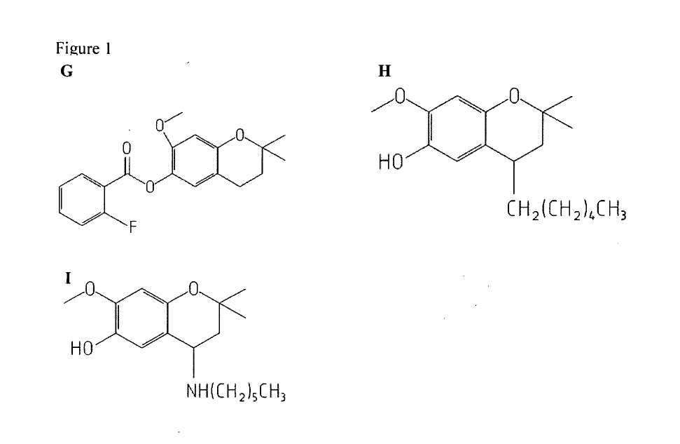

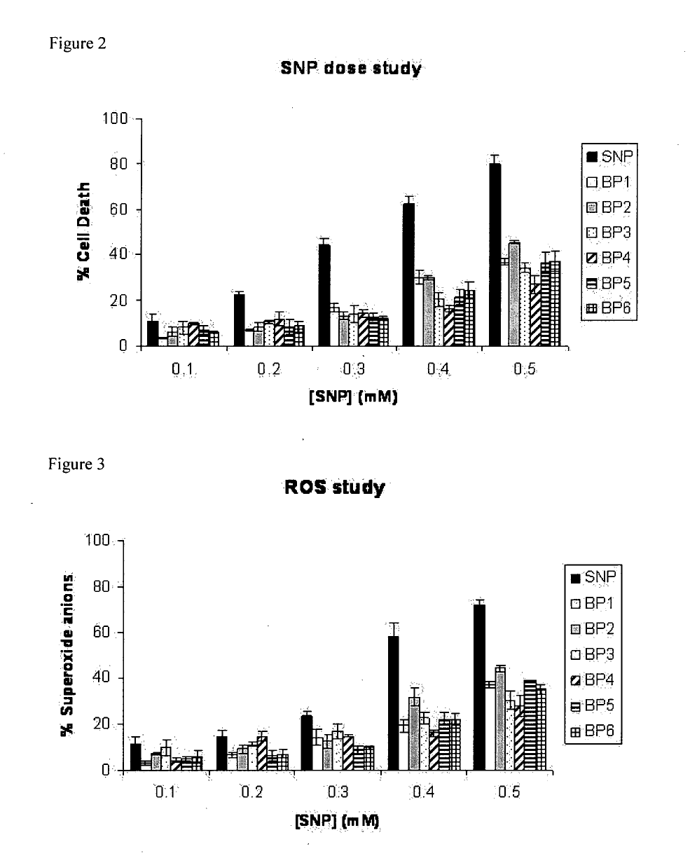

A solution of BP (1 equivalent) in anhydrous dichloromethane was added dropwise to a mixture of DCC (N,N-dicyclohexylcarbodiimide; 1 equivalent), DMAP (dimethylaminopyridine; 0.1 equivalents) and the corresponding acid in each case (i.e. palmitic acid for BP3 synthesis; 1 equivalent of acid) in anhydrous dichloromethane. The mixture was stirred for 3 h at room temperature. Formation of the corresponding BP derivative was monitored by thin layer chromatography. Next, the mixture was filtered to remove the appearance of the urea precipitate. The dichloromethane was evaporated under reduced pressure and the crude product was redissolved in hexane. The solution was kept overnight at 4° C. and filtered again the following day. Finally, the crude product was purified by column chromatography (silica gel, hexane: ethyl acetate 20:1) to obtain the pure BP derivative. The chemical structure of BP derivatives is shown in FIG. 1.

Ex-Vivo Methods

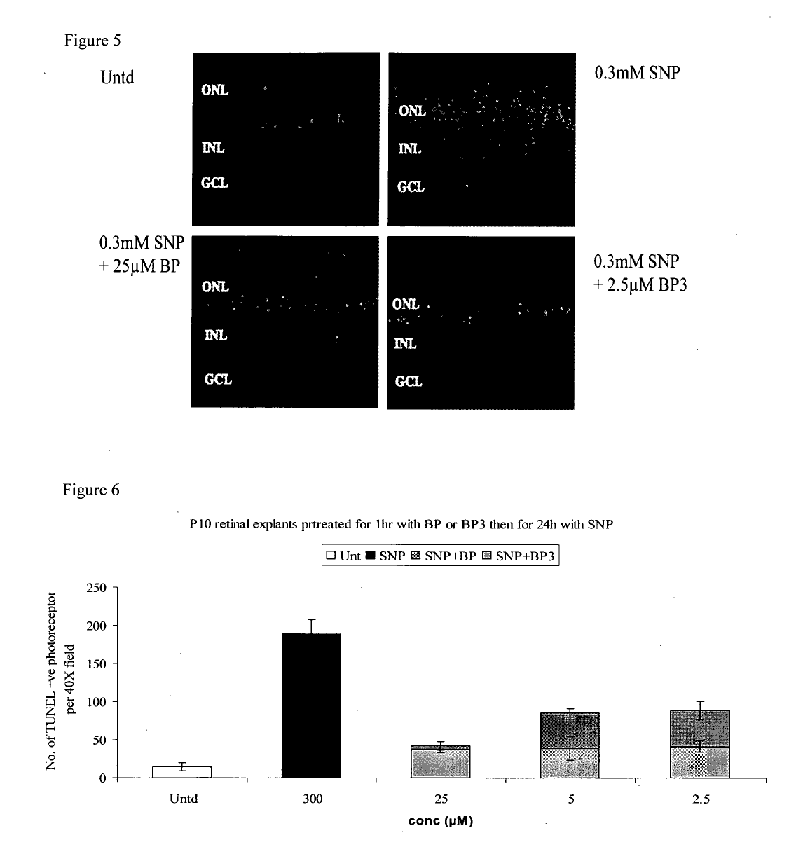

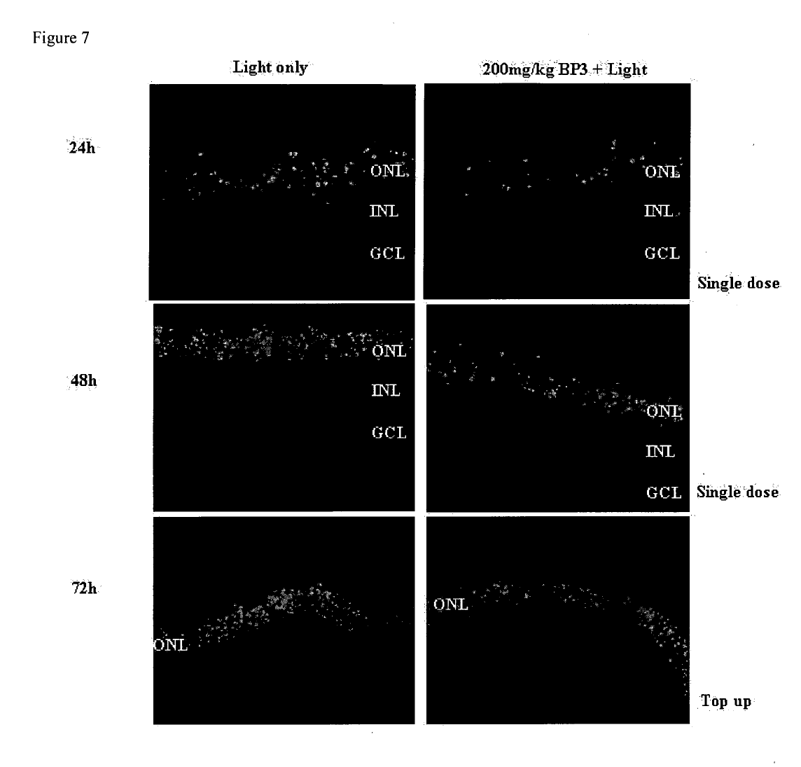

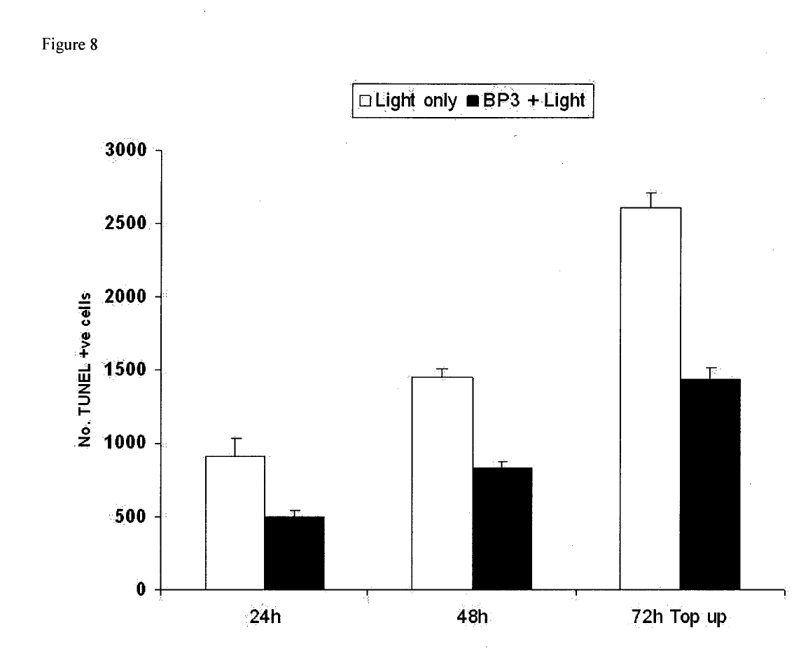

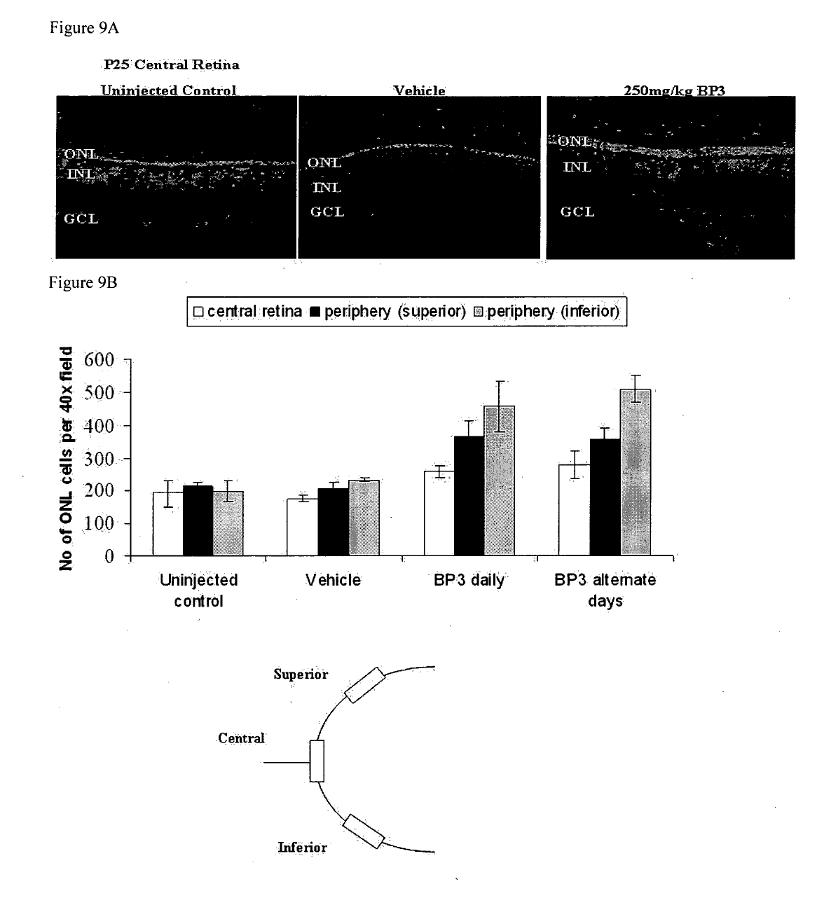

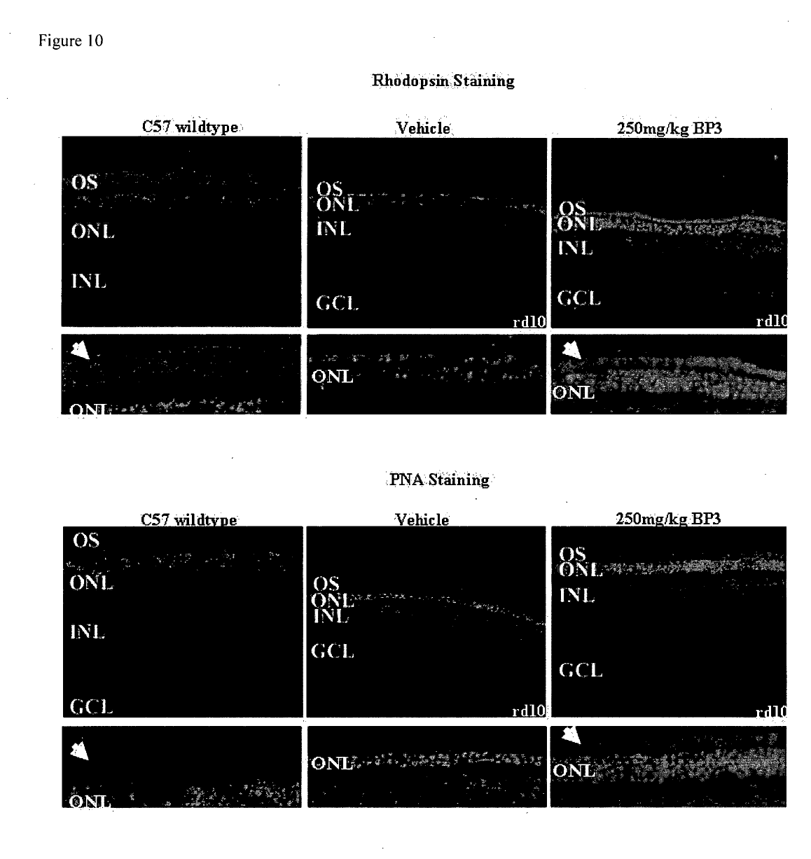

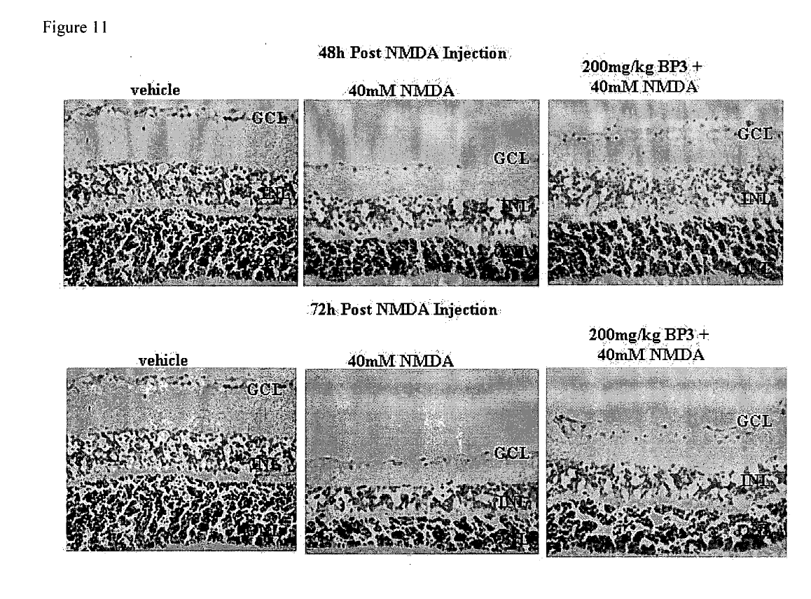

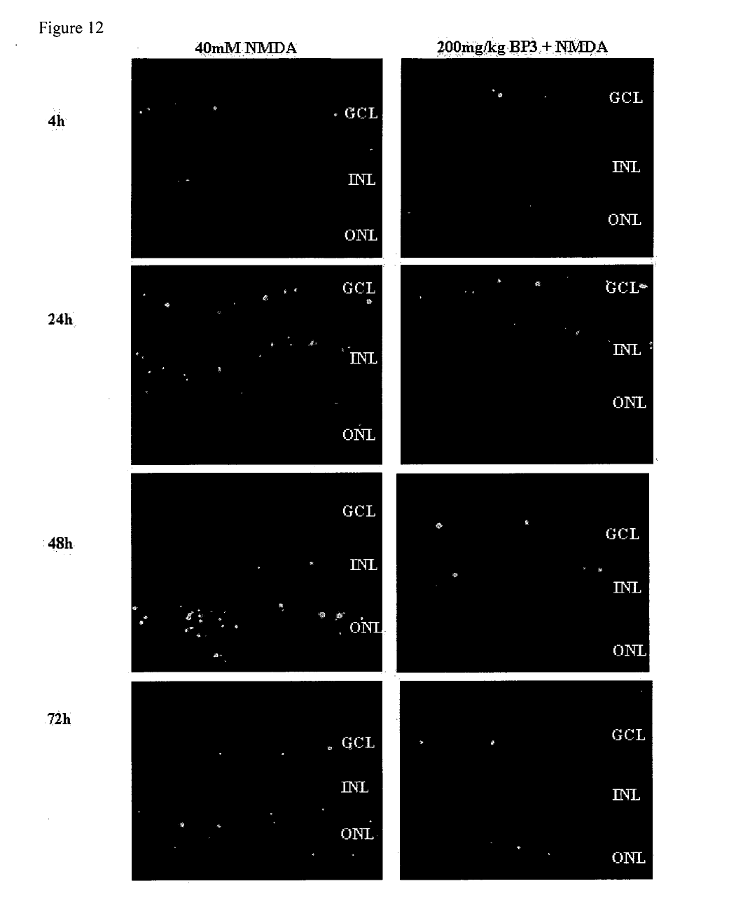

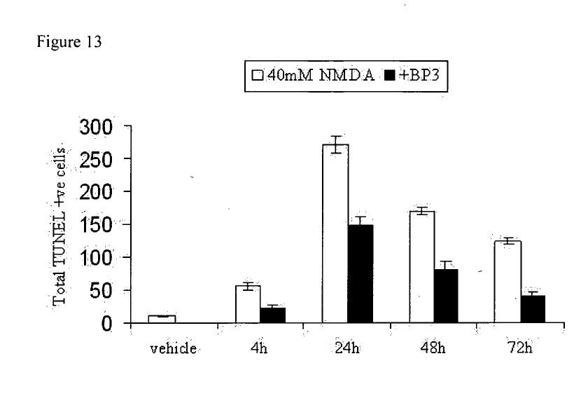

Retinal explant culture: Eyes from postnatal day 10, C57BL/6 mice were removed and cleaned with 70% ethanol. The anterior segment, vitreous body, and sclera were removed and the retina mounted on Millicell nitrocellulose inserts (Millipore, Billerica, Mass.) photoreceptor-side down. Explants were cultured without retinal pigment epithelium (RPE) in 1.2 ml of R16 specialised media (from Dr. P. A. Ekstrom, Wallenberg Retina Centre, Lund University, Lund, Sweden) without additional serum. Treated explants were cultured in medium containing 300 μM of the nitric oxide donor SNP (sodium nitroprusside) for 24 h. Pre-treatment with the Active was for 1 hour. FIG. 5 shows that photoreceptors are protected from SNP induced apoptosis by increasing concentrations of norgestrel.

Apoptosis detection by Terminal deoxynucleotidyl transferase-mediated biotinylated UTP nick end labeling (TUNEL): Retinal explants were fixed in 10% neutral buffered formalin overnight at 4° C., followed by cryoprotection in 25% sucrose overnight at 4° C. Frozen sections (7 μm) were incubated with terminal deoxynucleotidyl transferase (MSC, Dublin, Republic of Ireland) and fluorescein-12-dUTP (Roche, Lewes, UK) according to manufacturers' instructions at 37° C. for 1 h. Sections were mounted and viewed under a fluorescence microscope (Leica DM LB2; Leica, Nussloch, Germany) using an FITC filter. FIG. 6 shows that BP and a BP derivative, BP-3, protect photoreceptive cells from light damage in an ex-vivo retinal explant model, with BP-3 providing better protection.

Immunocytochemistry: Eyes were fixed in 10% neutral buffered formalin overnight at 4° C., followed by cryoprotection in 25% sucrose overnight at 4° C. Frozen sections (7 μm) were blocked with 0.1% bovine serum albumin (BSA) in 0.1% tween/PBS for 1 hour at room temperature. Sections were incubated with anti-rhodopsin antibody (LAB VISION Corporation, Fremont, Calif., USA) overnight at 4° C. Sections were washed and incubated with FITC conjugated secondary mouse antibody (Dako, Glostrup, Denmark) for 1 hour at room temperature. Following further washes, sections were mounted and viewed under a fluorescence microscope (Leica DM LB2; Leica, Nussloch, Germany) using a FITC filter.

Peanut agglutinin (PNA) staining: Eyes were fixed in 10% neutral buffered formalin overnight at 4° C., followed by cryoprotection in 25% sucrose overnight at 4° C. Frozen sections (7 μm) were blocked with 0.1% bovine serum albumin (BSA) in 0.1% tween/PBS for 30 minutes at room temperature. Sections were incubated with rhodamine conjugated PNA (Invitrogen, Dun Laoghaire, Ireland) for 20 minutes at room temperature as per manufacturers' instructions. Sections were mounted and viewed under a fluorescence microscope (Leica DM LB2; Leica, Nussloch, Germany) using a TRITC filter.

Hematoxylin staining: Eyes were fixed in 10% neutral buffered formalin overnight at 4° C., followed by cryoprotection in 25% sucrose overnight at 4° C. Frozen sections (7 μm) were stained in Hematoxylin (Sigma, Dublin, Ireland) for 10 seconds followed by a 15 minute water wash and 2-3 dips in acid alcohol. Following further washing, sections were placed in a 2% sodium bicarbonate (Sigma, Dublin, Ireland) solution for 30 seconds then dehydrated through an alcohol gradient. Sections were cleared in Histoclear (Sigma, Dublin, Ireland) for 5 minutes then mouted in DPX (BDH, VWR International Ltd., Poole, England) and viewed under a light microscope (Leica DM LB2; Leica, Nussloch, Germany).

In-Vivo Methods

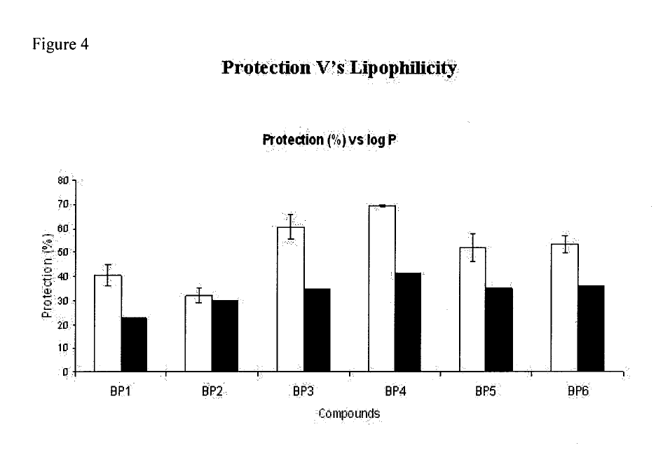

Light damage model: Balb/c mice were dark adapted for 18 h prior to exposure to constant light. Mice were injected intraperitoneally with the Active 1 hour prior to light damage. Immediately prior to light exposure their pupils were dilated with 0.5% cyclopentolate under red light. Retinal light damage was induced by exposure to 2 h of cool white fluorescent light at an illumination of 5000 lux. Following exposure to constant light, animals were placed in the dark for 24 h then killed immediately by cervical dislocation. TUNEL staining was performed as described above. FIGS. 4 and 5 show that 2 hrs light damage induces apoptosis after 24 hours in the ONL. Photoreceptors are protected by IP injection of 200 mg/kg of a BP derivative, BP-3.

Rd10 Model

The rd 10 mouse strain exhibits autosomal recessive retinal degeneration and has a point mutation in exon 13 of the Pde6b gene. It is a better model of the slow progression of typical human autosomal recessive RP than the acute light model as photoreceptor cells are lost over a period of weeks rather than days. Loss of photoreceptors in the rd10 mouse begins at approximately 2 weeks of age, with the peak of photoreceptor death occurring at postnatal day (P) 25.

Intravitreal Injections: Adult balb/c mice were anaesthetised using an intraperitoneal injection of ketamine hydrochloride 35-50 mg/kg (Pharmacia, Corby, Northamptonshire, UK) and xylazine hydrochloride 5-10 mg/kg (Chanelle Pharmaceuticals, Loughrea, Co. Galway, Ireland), and animals were placed in the pronate position. Injections were performed using a 5 μL syringe (Hamilton, Reno, Nev., USA) on which was mounted a 30-gauge cannula, and visualised using a binocular operating microscope. Using a 30.5-gauge needle (Becton-Dickinson, Drogheda, Ireland), an initial puncture was fashioned through the conjunctiva and sclera immediately posterior to the superonasal limbus. The cannula mounted on the 5 μL syringe was then introduced to the vitreous cavity through this opening, and directed backwards towards the optic nerve until the tip was easily visualised within the vitreous cavity behind the lens. NMDA (Sigma, Dublin Ireland) was diluted to 40 mM in PBS and 24 of solution (vehicle or NMDA) was slowly injected. The cannula was left in place for one minute then slowly withdrawn.

The invention is not limited to the embodiment hereinbefore described which may be varied in construction and detail without departing from the spirit of the invention.

1-22. (canceled)

23. A method for treating a retinal dystrophy comprising a step of treating an individual with a therapeutically effective amount of a compound of general formula (I) 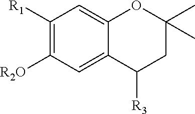
or a pharmaceutically acceptable salt thereof, wherein:
R₁ is a alkoxy, alkyl, ether or ester group;
R₂ has the formula 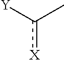
wherein Y is linear or branched, saturated or unsaturated, aliphatic group with from 2 to 23 carbon atoms, or a cyclic group, and which can contain substituents selected from the group consisting of hydroxyl, alkoxy, amino, carboxyl, cyano, nitro, alkylsuphonyl or halogen atoms, X is O or S; and R₃ is any substituent.

24. A method as claimed in claim 23 in which R₁ is an alkoxy and R₃ is H.

25. A method as claimed in claim 23 in which R₁ is methoxy and R₃ is H

26. A method as claimed in claim 23 in which 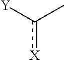
is selected from the group consisting of: tert-butanoyl; hexanoyl; 2-ethylhexanoyl; octanoyl; decanoyl; lauroyl; myristoyl; palmitoyl; stearoyl; oleoyl; or lineoyl.

27. A method as claimed in claim 25 in which Y is an alicyclic group, or an aromatic cyclic group, or a heterocyclic group.

28. A method as claimed in claim 23 in which, 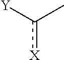
is selected from the group consisting of: —CO—(CH₂)₀₋₆phenyl; —CO—(CH₂)₀₋₆(1-napthyl); —CO—(CH₂)₀₋₆(2-napthyl); —CO—(CH₂)₀₋₆CH(phenyl)₂; —CO-(2-fluorophenyl); —CO-cyclohexyl; α-lipoyl; L-prolyl; D-prolyl; biotinyl-CO-(4-imidazolyl); —CO-(2-pyridyl); —CO-(2-thienyl); —CO-(2-furyl); and —CO-(3-furyl).

29. A method as claimed in claim 23 in which X is O.

30. A method as claimed in claim 23 in which R₁ is methoxy and OR₂ is selected from an acetate ester, a pivalate ester, a laureate ester, a 2-methylhexanate ester, a phenyl ester, and a o-fluorophenyl ester.

31. A method as claimed in claim 23 in which the compound of general formula (I) is 3,4-dihydro-6-hydroxy-7-methoxy-2,2-dimethyl-1(2H)-benzopyran.

32. A method of claim 23 in which the compound of general formula (I) is a compound of general formula (III), 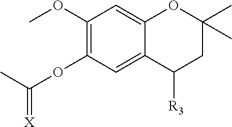
or a pharmaceutically acceptable salt thereof, in which X is O or S, and R₃ is any substituent.

33. A method as claimed in claim 23 in which R₃ is selected from the group consisting of: H; halogen; lower alkyl; lower alkoxy; hydroxyl; amine; thiol; NHR4; or a substituted or unsubstituted aromatic ring structure in which the substituents (if included) are selected from the groups consisting of H, halogen, lower alkyl, lower alkoxy, hydroxyl, amine, and thiol, and wherein R₄ is any substituent.

34. A method as claimed in claim 33 in which R₄ is selected from the group consisting of: halogen; lower alkyl; lower alkoxy; hydroxyl; amine; thiol; or a substituted or unsubstituted aromatic ring structure in which the substituents (if included) are selected from the groups consisting of H, halogen, lower alkyl, lower alkoxy, hydroxyl, amine, and thiol.

35. A method as claimed in claim 32 in which R₃ and R₄ are, independently, C4 to C8 straight alkyl chains.

36. A method as claimed in claim 35 in which R₃ and R₄ are, independently, C5 to C7 straight alkyl chain and ideally a C6 straight alkyl chain.

37. A method as claimed in claim 36 in which R₃ and R₄ are, independently, C6 straight alkyl chain.

38. A method as claimed in claim 23 in which the compound of general formula (I) is administered to the eye.

39. A pharmaceutical composition formulated as a solution suitable for local delivery to the eye, the composition comprising a compound of general formula (I) 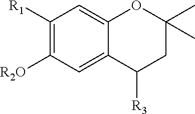
or a pharmaceutically acceptable salt thereof, wherein:
R1 is a alkoxy, alkyl, ether or ester group;
R2 has the formula 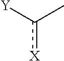
wherein Y is linear or branched, saturated or unsaturated, aliphatic group with from 2 to 23 carbon atoms, or a cyclic group, and which can contain substituents selected from the group consisting of hydroxyl, alkoxy, amino, carboxyl, cyano, nitro, alkylsuphonyl or halogen atoms, X is O or S; and R3 is any substituent.

40. A pharmaceutical composition as claimed in claim 39 in a form selected from the group consisting of: eye-drops; solution suitable for intraocular injection; and solution suitable for intraocular injection.

41. A pharmaceutical composition as claimed in claim 39 in which R₁ is an alkoxy and R₃ is H.

42. A pharmaceutical composition as claimed in claim 41 in which R₁ is methoxy and R₃ is H.
